(19) United States
(12) Patent Application Publication
(10) Pub. No.: US 2013/0253375 A1
(43) Pub. Date: Sep. 26, 2013

(54) Automated Method Of Detecting Neuromuscular Performance And Comparative Measurement Of Health Factors

(71) Applicant: Henry Nardus Dreifus, Sanford, FL (US)

(72) Inventor: Henry Nardus Dreifus, Sanford, FL (US)

(21) Appl. No.: 13/425,699

(22) Filed: Mar. 21, 2012

(51) Int. Cl. A61B 5/103

(52) U.S. Cl. 600/587

(57) ABSTRACT

The invention described here enables the real-time, low-cost, non-invasive measurement of neuromuscular performance for numerous healthcare and screening applications including assessing maladaptation prediction via a screening session as well as provision the application of correlated and corrected measurements of force displacement. The tests to be performed and subsequently measured are dynamically determined and administered using a computer guided and prompted process to acquire and process a subject's performance including predicting future injury. The system as well is adaptive and allows the introduction of new tests, or streamlining and combination of performance tests based on acquired data across the universe of screening platforms.

### FIELD OF THE INVENTION

This invention encompasses embodiments for multi-dimensional automated computer-guided neuromuscular performance measurement of various aspects of human function including lower limbs and lower back to provision early detection screening and prediction of health performance related issues. Said invention as well can provide real-time diagnostic feedback to healthcare professionals such as for the screening, detection and treatment of limb maladaptation, and predicting propensity for injury or other neuromuscular aspects.

### BACKGROUND OF THE INVENTION

This field of invention is related, but not limited, to the computer-guided automated real-time measurement of select neuromuscular performance based on observed detection of parameters related to the specific performance of particular tasks to provide an immediate computed result of measured movements compared against an adjusted and normalized predicted baseline value set adjusted for such variables as gender, age, weight, prior injury, and other aspects. Human beings and many other living species exhibit bi-lateral symmetry, which is commonly expressed in limbs, eyes, ears, and other anatomical aspects. Although bi-laterally symmetric, there are subtle, and at times more pronounced, differences such as in limb length, strength, agility and size. An individual may also have a predisposition or preference for a dominant limb (e.g. right handed, left handed), which is also exhibited in the legs (right legged, left legged, etc.). Small differences over a lifetime can cause a maladaptation in the course of use of the limbs due to congenital, experiential (e.g. injury) or environmental (e.g. a baseball pitcher, football place-kicker, repetitive work in a factory, etc.) factors which over time can exert pressure across the muscular-skeletal system and result in uneven wear, pressure and eventual injury. The scope of this invention is to develop a process to measure the potential and early screening for maladaptation, as well as to provide longitudinal measure of correction (e.g. through recognition and treatment, physical therapy, or other corrective interventions).

Preventing injury and early detection of health issues have been one of the most significant contributors to increasing life expectancy and longevity. Screening for early detection has made a major impact on both the quality and quantity of life. Such diagnostic tests for eyesight, serum glucose levels (for diabetic screening), hearing, blood pressure, serum cholesterol for atherosclerosis, Papanicolaou tests (pap smears) and gamma-seminoprotein, or prostate-specific antigen (PSA) levels etc. (for cancer), have and continue to save lives and increase longevity. As lifespans increase so do the health risks, including the risk of falling and breaking bones as one gets older. A major contributing component to this risk is the subtle but chronic adjustment(s) or adaptation(s) to limbs and limb performance over time. This is not only for the aged population, but as well may begin to be expressed in children, and can have implications to predisposition to injury, such as in school sport activities. Providing non-invasive methods to improve the early detection, to screen for propensity for injury—including injury risk prediction—will improve the ways to mitigate the long term impacts due to maladaptation resulting in lower back and limb pain/discomfort. Through such early detection, preventative measures such as introducing corrective exercises, appliances such as orthotics, specialized sleeping systems and corrective clothing can be implemented to adjust for maladaptation and improve the overall net health outcomes as well as mitigate potential for downstream injury.

This invention also can help in providing risk assessments prior to engaging in sports to prevent injury, or re-engage in sports after an injury, to prevent early return to play that could cause long term harm. The toolsets driven by this invention can also propel more precise and accurate protocols for designing personalized treatment plans and provide continuous assessment and immediate feedback of degrees of improvement due to the provided therapy intervention(s). A byproduct of this invention will also allow for the objective measurement and assessment of degree of disability, which could help in determining if an individual would be able to safely return to work, or an athlete safely return to the field to play a sport.

Present and prior art and techniques teach both invasive and non-invasive measures of performance including direct isokinetic measurement of specific muscles and muscle groups, range of motion, strength, endurance, etc. While these tests are presently invaluable in the diagnosis and treatment of orthopedic conditions, they are time consuming, expensive and labor intensive. The protocols require potentially hours of parametric testing with additional analysis and processing of the data which can take weeks or more to provide a diagnostic result. Moreover, the subject/patient is subjected to a number of repetitive and strenuous testing (testing to fatigue, maximum strength displacement, etc.) that in the case of recovering from an accident or surgery can introduce additional pain, discomfort, and potentially create or aggravate an injury.

Implementations of force displacement measurement are well known and have been practiced for measurements in industrial applications including weight and balance and for displacement analysis. These measurement platforms include products by the Mettler-Toledo Corporation, MTS Corp. and others which manufacture precision sensors and equipment for calculating mass and weight distributions, etc. Prior art in Mooney U.S. Pat. No. 7,946,928 and others including Heisler U.S. Pat. No. 5,150,902, Osmudsen U.S. Pat. No. 6,616,556 and Berme U.S. Pat. No. 6,389,883, for the purpose of balance/tremors measurement using force measurement, teach the measurement of “foot forces” for a golfer using a force measurement technique. These prior implemented approaches teach the need to measure absolute displacement and lateral forces. Huberti in U.S. Pat. No. 5,042,504 and Confer in U.S. Pat. No. 4,745,930 design approaches that isolate the measurement of a single function across a specific test regime. The sensing measurement can either be in the sole of a shoe or using an external force measurement platform. The present invention improves upon these techniques in a number of ways including integrating the measurement of rates of acceleration (and deceleration) as well as other stability and de-stability measures. Further, through the enhancement of predictive algorithmic correction, such as Kalman filtering across the sequence of measurements, can improve the fidelity and correct for inaccuracies in the acquisition and measurement of a specific test sequence. This correction supports the natural variation and other factors in the testing process including the measurement of the calculated total force development/displacement (strength) and landing stability and other metrics.

Force measurement has been applied in training/retraining as taught by measuring force movements for actuating robotic limbs including legs as exemplified in US Patent Publication 2010/0324699 by Koeneman and measuring methods developed, such as taught by Nashner in U.S. Pat. Nos. 4,738,269, 5,980,429, 6,010,465, 6,190,287, 6,632,158, 7,127,376 and publications 2007/0093989, 2004/0127337, which categorize the movements and conformance based on baseline position and static measurements from equilibrium. The prior art teaches stability and degree of adaptation to equilibrium, as opposed to measuring rates of destabilization, including the rates of force development and acceleration away from equilibrium. In effect the present invention provides a counter intuitive approach through measurements of instability (movement and action) including calculations of acceleration and rates of force development as well as landing forces (e.g. rates of deceleration and how adaptation is measured to achieve stability).

Additional prior art teaches “in motion” analysis including video imaging such as described by David in U.S. Pat. No. 6,816,603 that analyses the assessment of gait and gait adaptations. This approach teaches the detection of variation, but does not yield a quantitative assessment. Video-based analytics have as well the drawback of masking other pathologies. Other approaches, including direct force measurements sampled over periods of time as taught by Frynkman in U.S. Pat. No. 7,455,620, have the disadvantage over the present invention in the need for long data acquisition cycles and sustained performance. Further, Frynkman requires the implementation of force measurement platforms positioned in a single dimension; as described this approach requires two separate measurement platforms placed underneath a treadmill which are mechanically decoupled. Further, this only allows the measurement of vertical displacement, or rate of force development, and does not also focus on the analysis of rates of acceleration (and deceleration) which equates to measurement of rates of changes in stability and de-stability in the generation of force under neuromuscular control. Further this art teaches the need for use of more than one force measurement platform simultaneously across a single test regime.

Other approaches, including the work described by Otto as published in 2010/0076563, outlines building an understanding and computer simulation of a specific maladaptation based on various positional measurements coupled with force platform displacement observations. The stated purpose is to introduce a prospective limb implant and to predict the potential improvement and outcome based on the force measurements. This approach, similar to the isolated performance analysis of other prior art, teaches the focus on a single element, e.g. a knee and associated elements (e.g. ligaments, etc.) but fails to analyze the degree of the entire neuromuscular adaptation (and resulting compensations) across the entire neuromuscular portfolio and the additional challenges of adaptation across all biologic components that comprise the neuromuscular “system” of inter-connected components.

### SUMMARY OF THE INVENTION

To provide an enhanced understanding beyond a static examination by a health professional of the limb performance and determination of the degree of adaptation (or maladaptation), the present invention provides a number of dimensions for non-invasive, real-time measurement of individual and combined elements of the spectrum of limb performance such as range-of-motion, rate of force development, degree of balance, rate of landing force absorption, symmetric and asymmetric measures, level and non-level baseline, and others. The present invention improves upon prior art in a number of ways including establishing a dynamically programmatic protocol via establishing a determining process to indicate the proposed set(s) and sequencing of tests for measurement of force development and stability parameters application of multiple tests, or the reduction or elimination of tests that are not necessarily relevant to enhance the neuromuscular analysis. As well in the case of repeated, longitudinal testing, the subject may “learn” and anticipate tests and as such become trained to the expectation to perform the test sequence. This could result for example in mischaracterizing the ‘landing’ or stabilization deceleration forces. This invention seeks to provide the most accurate measurement quality. Further, especially in the case of injured or disabled individuals due to their condition, they may become exhausted over the course of administering multiple tests, especially when conducting fixed protocols. This may as well mask or mitigate the accuracy of the testing and results, thus providing a less than optimal assessment. As this invention looks to determine such measurements including rates of stabilization and rates of destabilization, the purpose is to measure maxima of force efforts as opposed to static strength measures. Thus the importance of determining the maximum rates of force development, force absorption and other measures not solely in terms of absolute maximum and minimum values, the rates of development (acceleration and deceleration) are as well important metrics to acquire as a part of the testing process.

Multiple modalities of test are performed and measurements of specific neuromuscular tasks as evaluated by taking direct measurements via a local processor which are combined and integrated to determine specific measures of adaptation coupled with adjustments of limb activity by a computer guided protocol that both sequentially and via integrated processing can calculate rates of force development, stability (including lateral, individual and combine limbs) and rate of force absorption—such as landing forces adjusted for factors including gender, age, and other factors such as prior injury or known diagnosis or pre-existing conditions.

In addition, this invention can detect predicted risk of injury based on correlation of derived data from a set of at least one measurement means. The process to derive the tests needed to be conducted and the measurements performed may be variable based on the computation of a set of at least one of the context of the testing, and other information which can include a number of factors such as those dynamically calculated based on an understanding of the observed measurements, applied protocols and other such information including the age, gender, weight and other factors. This determination of the types of tests and testing protocol is derived and may be different across test subjects based on said measurements and other information.

Specific individual tests can be further combined to develop at least one set of derived measurements that could include the comparative analysis (including such analysis as inter-limb variation). This approach allows for the present invention to develop an understanding of the underlying conditions including the nature of the adaptive process to measure the degree of force generation, and resulting compensating processes that can be used to calculate the degree of maladaptation of the resulting neuromuscular system. The integrated analysis across the determined test functions performed allows the invention to further the ability to determine which of the component elements of the neuromuscular system are being most impacted and therefore aid in the diagnostic screening and assessment of other indicated conditions. A maladaptation is usually manifested across more than one component of the neuromuscular system.

The present invention is further enhanced through the combining of at least one of a set of tests that are determined by the entered information and tests performed, which could include prior tests conducted to establish both a baseline and longitudinal comparison over time, such as during a rehabilitation course of treatment. This improves upon the prior approaches which require the discrete measurement, including specific neuromuscular component isolation, that whilst useful, fails to provide the full understanding of the neuromuscular system and the specific adaptation (or maladaptation). The computed determined tests are based on the evaluation of the specific factors which may be related to, but not necessarily fixed in association with, attributes including gender, age and other factors. In the case of gender, there are noted differences including the proportional ratios of limb and limb elements, center of gravity (based on distribution of mass, and therefore differences in center of mass), height, bone density (such as exhibited after child bearing which may result in bone loss) and other factors. These factors can impact neuromuscular performance, and more specifically the resulting adaptations. Further, the chronic effects of osteoporosis, including an increased propensity to and risk to bone damage, breakage and other trauma can drive adaptive behaviors. Such factors are therefore relevant in the determinant process and have subsequent bearing in the integrated analysis beyond the determining protocols.

Integrating the measurements provides multiple benefits to the invention. This allows the diagnostic advantage to concomitantly improve the fidelity of the individual measurements as well as to provide a means to derive converging diagnostic (pre-)screening results. The process of performing a set of distinct and discrete tests is to yield data that isolates and predicts individual neuromuscular pathology(ies) as well as to integrate and combine to build an understanding of the net or combined neuromuscular adaptation (or maladaptation). Typically there are multiple corrective forces attempting to compensate a maladapted component, such as results from a direct injury or through congenital or hereditary condition, repeated injury or non-corrected limb function. Over time these multiple corrective forces in themselves may contribute to the introduction or exacerbation of maladaptation and may not be properly detected or predicted. Through the understanding of the multiple dimensions of measurement this detection, including the predicted predisposition to maladaptation, can be calculated. Through measurement across known and expected correct adaptation, the degree of variance across the dimensions can be analyzed, including a correlation of the proximity to the introduction of the underlying cause of the maladaptation, such as the timing of when an injury occurred, to the corrective protocols including surgical and physical therapy interventions, etc.

Integrating the individual specific tests also improves the underlying data fidelity and supports the diagnostic confirmation of the other conducted and subsequently correlated individual tests. These measures can also be applied to direct specific algorithmic post processing including data filtering to refine and to converge individual test analyses to improve the net accuracy. As each test and test subject is performing a series of individual specified computationally calculated determined tests the resulting outputs can be used to provide analysis to predict the acceptability and resulting quality of the anticipated test and test result. This can be used to determine if a specific test should be repeated (an unacceptable anticipated quality measurement due to the test subject incorrectly performing the indicated specific neuromuscular test) by providing a mechanism to computationally correct (such as applied by error correction techniques as taught across numerous applications from video and audio coding) and other methods such as applied by Kalman filtering or a hidden Markov, Bayesian or other such mathematical approaches. This allows for the improvement of the quality of test data as well as minimizing the number of needed tests to be performed or repeated. This improvement allows for both the increase in underlying data quality as well as the reduction in the quantity in the number of tests necessary to evaluate a specific diagnostic condition.

The present invention also incorporates a control program that self-calibrates, develops, directs and integrates not only the determination of the testing and testing sequence process, but as well verifies the requested test performance and subsequently processes the test measurements using both individual and combined measurements as described below. This control program also supports prompting by the computer to guide the test subject to perform the indicated test and shows acceptance of the data to be evaluated and subsequently provide the measured results. The control program also determines not only if the test was performed correctly, but as well assess if a test needs to be repeated, or if the data is of sufficient fidelity that the measurement can be corrected through the computational assessment such as filtering and other programmatic analytic techniques applied to the underlying individual measurement and previously acquired data. The control program also evaluates and maintains the underlying data values, testing protocols and measurement methodologies to process and determine if additional tests may be needed due to either uncertainties in the already acquired measurements, or the computed decision that one or more tests does not correctly correlate to anticipated or expected value ranges and therefore should be repeated. This control protocol also provides the ability to support the potential to enable the self-administration of the tests, such as may be used by a sports athlete or in a sports training facility.

Additionally this invention contemplates gaining additional understanding based on comparative analysis of prior tests both on a longitudinal basis such as a time series of tests conducted on a periodic basis as well as on a comparative basis against comparable similar populations of tests performed across confirmed diagnoses such as an adjustment for age, nature of injury, injury status, diagnostic determination (such as represented by medically accepted diagnostic related group (DRG) codifications), gender and other factors.

The approach of the present invention overcomes a number of limitations of traditional diagnostic methods including invasive methods by integrating across a set of tests and providing comparative (including longitudinal and associative population referential comparatives) in a single measurement platform that conducts a sequence of measurements that does not require intensive sustained physical exertion such as used in an isokinetic evaluation, and provides for immediate diagnostic/screening results and feedback.

Through the use and concurrent evaluation of several variables across the testing protocol, this assessment approach provides for concurrent error measurement and correction and as well calculating converging insight into various diagnostic conditions. A series of small tests offers stakeholders the opportunity for early detection and prevention of preventable injuries caused by prolonged maladaptation through an analysis evaluated across the set of specific subject measurements as well as time series measurements and comparable population measurements. Further, the present invention will allow stakeholders including medical professionals a non-invasive longitudinal tool to aid in the diagnostic treatment of chronic or congenital maladaptation, as well as support rehabilitation and specialized training regimes for specific purposes, such as professional athletics, military personnel and others. The invention further lowers the cost of testing using a single force measurement input device as well as automated error correction to decrease the testing times and improve the fidelity of the measured results.

The invention presented here enables the real-time, low-cost, non-invasive measurement of neuromuscular performance for applications including assessing maladaptation prediction via a screening session as well as the application of correlated and corrected measurements of force displacement. The tests to be performed and subsequently measured are dynamically determined and administered using a computer guided and prompted process to acquire and process a subject's performance. The system is as well adaptive and allows the introduction of new tests, or the streamlining and combination of performance tests based on acquired data across the universe of screening platforms.

### BRIEF DESCRIPTION OF THE DRAWINGS

FIG. 1 is a high-level overview of the major components of the system.

FIG. 2 is a high-level flow diagram of the component processes for the measurement and data acquisition.

FIG. 3 is a high-level flow representation of the measurement processing and analysis performed within this application of the present invention.

FIG. 4 is a block diagram of the associated software processing elements comprising the analysis modules incorporated within the real-time analysis portion of the system presented including the multi-factor/multi-measurement calculations.

### DESCRIPTION OF THE EMBODIMENTS

The preferred practice of a real time neuromuscular screening and analysis tool is based on a simplified and streamlined approach through the acquisition of specific dynamically computed and determined measurement criteria established through building an understanding based on pre-existing data such as input into the system. The preferred embodiment is based on a network based solution that is interconnected with a back-end database and data processing framework that continuously improves the fidelity, depth and breadth of measurement, testing and evaluation.

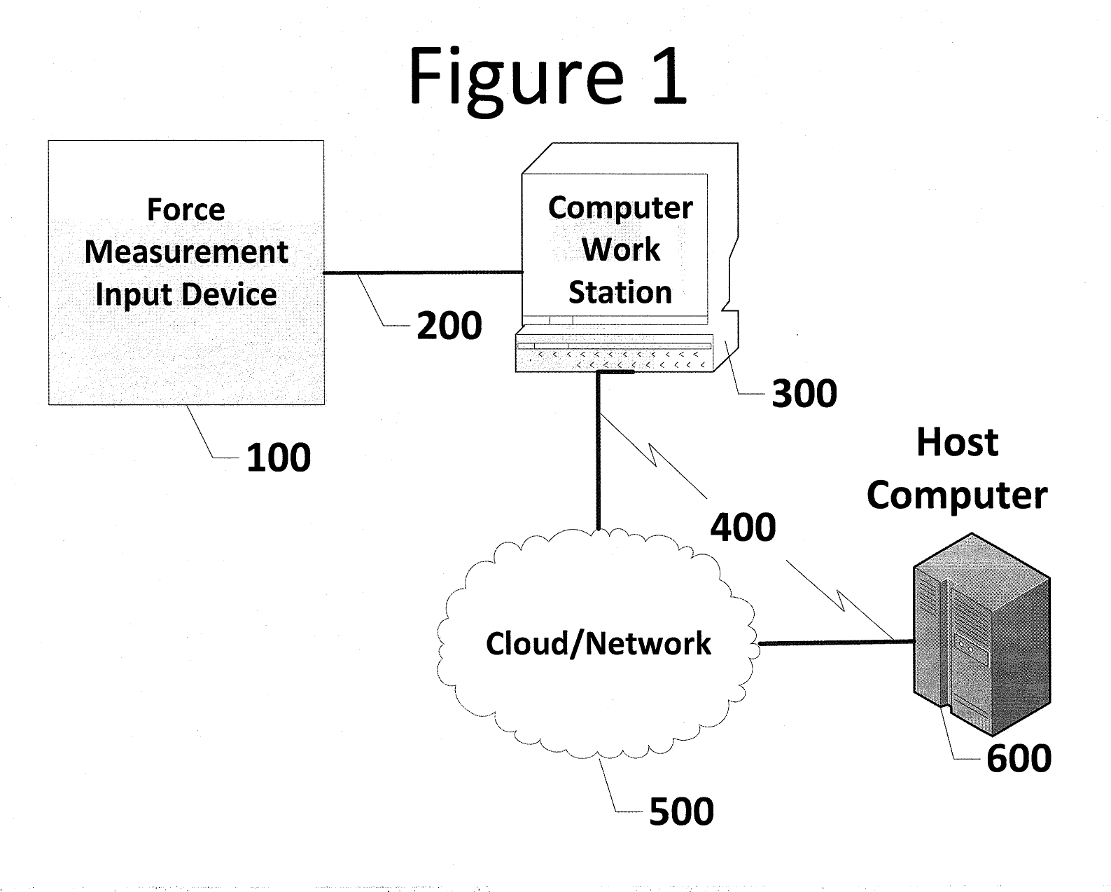

The embodying system summarized in FIG. 1 consists of a force measurement data acquisition device (100) such as a force plate. These are manufactured by a number of companies including Bertec Corporation, located at 6171 Huntley Road, Columbus, Ohio, AMTI Corporation, located at 176 Waltham Street, Watertown, Mass. and others. Products based on such platforms including NATUS' located at 1501 Industrial Road, San Carlos Calif. products are based on strict protocol based scripted testing protocols. These products focus on measurement and assessment of motor control and balance. The force measurement device is connected via a data cable (200) to a local computer work station (300) that provides for an integrated display, input device and optionally a printer or output device. This is connected via a wired or wireless connection (400) through a network (500) such as the internet or private network to a back-end host computer (600) which maintains the underlying databases and supports back-end processes including the ongoing analytic analysis and acceptable measurement values, etc.

The preferred embodiment of the invention is designed to be operated either directly by the individual undergoing the test (self-testing) or with a trained administrator without the need for an advanced medical or physical therapy degree, training or certifications. The computer guided approach generates not only the specific tests that need to be performed, but as well can provide for a real time analysis including an assessment if each of the measurements was correctly conducted and the data is usable. This automated validation and verification mitigates potential “subjective” assessment by a test administrator, and through applying a consistent machine evaluated validation, a more reliable and repeatable result. The heart of the system lies in the software platform and cumulative underlying and continuously improving reference data base which determines the test regime as well as real-time processing and output of the test results.

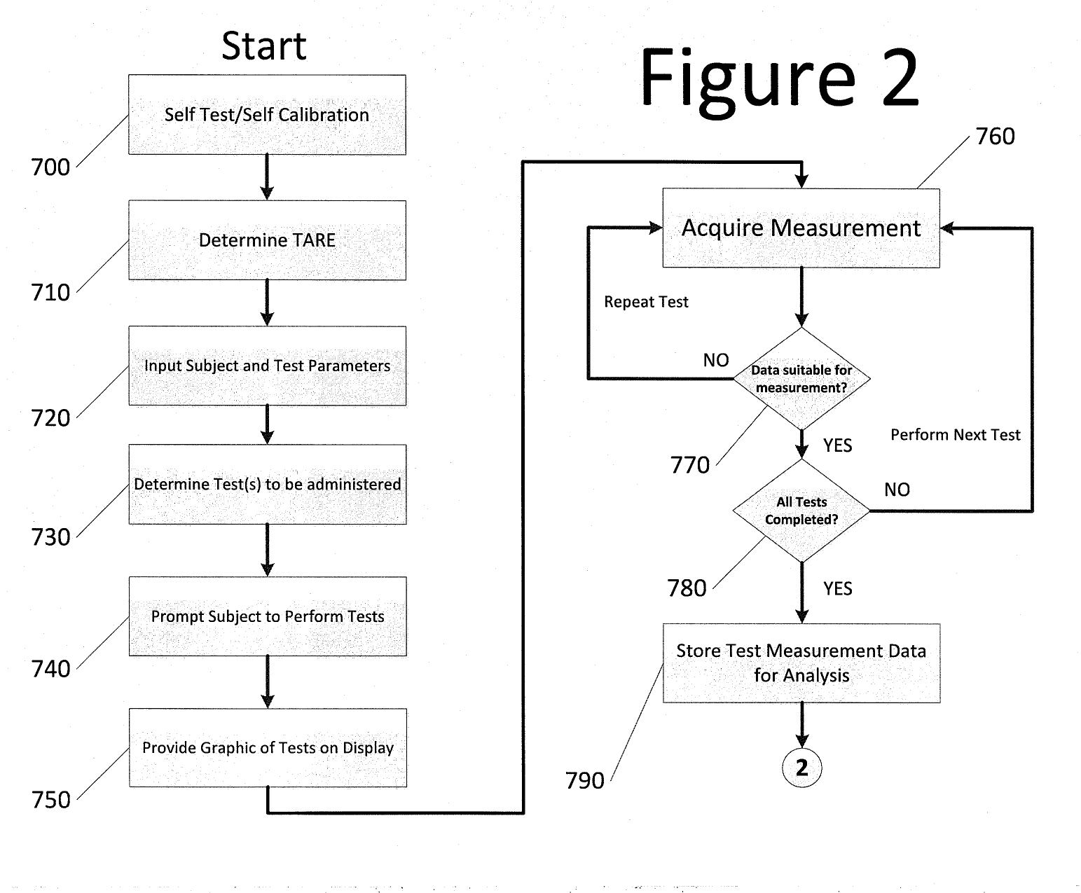

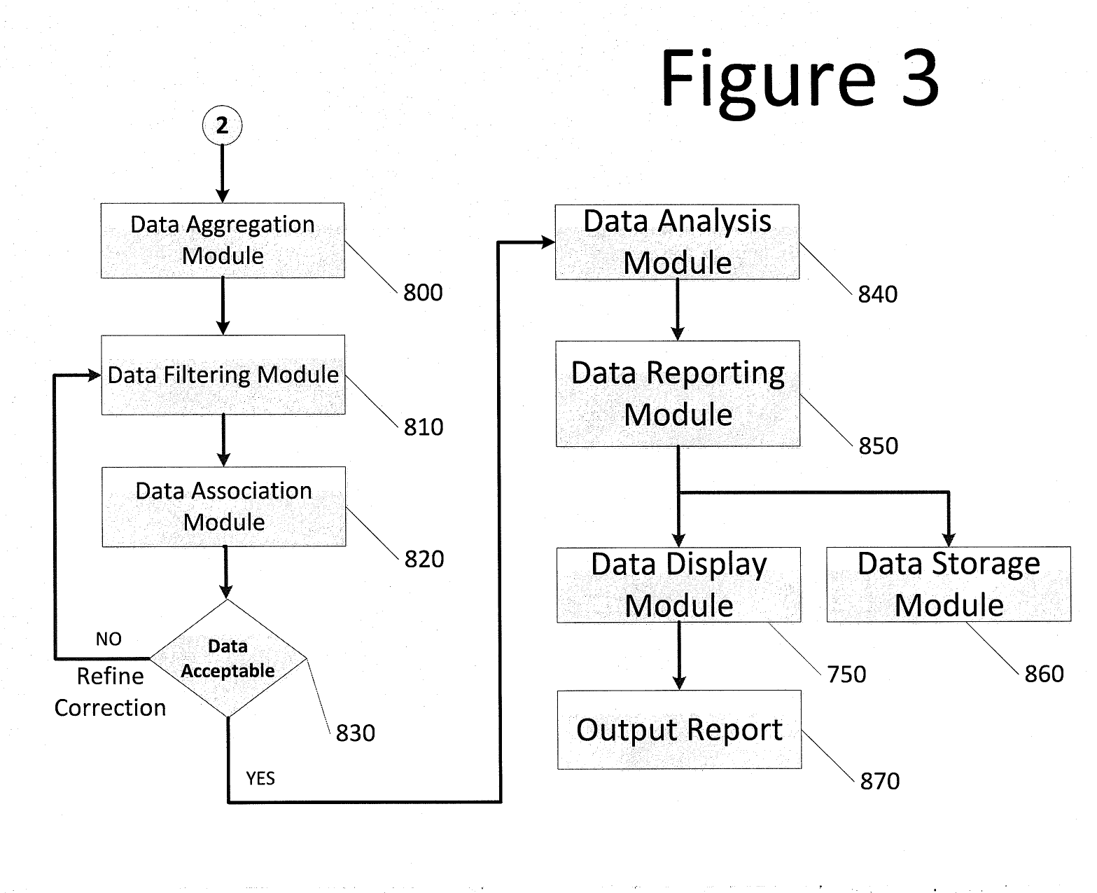

The system programmatically guides the conduct of the testing process as summarized in FIGS. 2 and 3. In FIG. 2, the computer workstation (200) first performs a series of self-tests of the detection and integrity of the measurement platform (100). These self-tests (700) are designed to assure the component and system integrity. After the self-calibration, the measurement platform measures and calculates the zero weight (710) or tare to set the baseline for subsequent measurements. An operator then enters specific subject information (720) on the computer workstation (200) to include basic information such as patient's name, and other parameters to include gender, confirmed diagnoses, age, and other factors. Subject weight is determined by the measurement platform. Information is collected and processed and correlated with the back end host computer (600) via a data communication network (400) via a network connection (500) to extract relevant information such as prior test results or adjacent comparable data to develop a determination of which tests to perform.

Based on the information provided and derived, including data coupled with the pre-established testing protocols the computer workstation (730) will calculate which functional tests need to be performed and measurements taken. The result is a task list of test measurements that are based on the determined measurements needed to complete the functional analysis. This then prompts (740) the subject and as appropriate a proctor accompanying the subject (such as a test subject who is recovering from an injury, or is a juvenile, aged, etc.) On the workstation (300) co-located to the measurement input device (100) there is a graphical depiction of each of the requested measurement procedures provided (750), such as lifting a leg, stepping onto the measurement input device, etc. There is no specific sequence of tests that need to be performed, only that the tests determined are acquired. In the case that a certain test cannot be acquired, there is a menu section on the workstation to indicate that a specific test is omitted from the data input. The subject then is prompted to perform the tests determined in (730) and to begin the testing in (760). This is an iterative process. Upon acquiring the data the work station assesses the input data measurement against a number of filtering and other criteria in (770). This includes potential comparisons (if existing) to prior tests conducted on the same test subject, as well as tests performed in similar populations to include age, gender, specified diagnosis (such as a DRG) and other factors. Additionally the measurement input is subjected to range testing and other parameters to assure correct input measurements are being acquired, confirm correct operation of the measurement input device (e.g. detect input device failure), and other parameters including maximum deflection detection (or over-range errors). The decision criteria are dynamically established for each of the computer prompted/guided measurement steps, and therefore each individual measurement is subject to its own acceptance parameters.

Upon acceptable measurement acquisition the information is stored in raw format along with other data to include the test success factor raw score as determined in (770) and other data as appropriate. In step (780) the work station (300) determines if there are still additional tests to be completed, and returns to step (760) to request the subject to perform the next measurement input. Once all the tests are completed the computer work station stores the results and supporting parameters in (790) and begins evaluating and processing the measurement data both individually and collectively.

The Data Aggregation Module (800) accumulates the raw test data and score supporting parameters (to include conformance and acceptance calculation) to associate and determine absolute maxima and minima boundary force displacement information as well as to provide measurement filtering to remove noise and outlier non-conforming measurements. The data filtering step in (810) improves the basic information captured in the measurement acquisition step (760) to isolate specific sub-component information including determining rates of force development, rates of acceleration (such as take-off forces), degrees of stabilization (landing forces) and times of stabilization measurements including initial and computed steady state stabilization. The association platform (820) correlates similar test evaluation parameters and calculates anticipated correlated factors across all test measurements. In step (830) data is subjected to a series of data correction regimes in search of an optimized “fit” of all test performance data to detect, and as appropriate correct to maximize the data fidelity across all measurements. A resulting output is submitted to the data analysis module (840) which includes the raw data measurements as well as the corrected data and algorithms supplied. It is possible that more than one correction vector can be applied across a set or subset of the data processed in the prior steps.

The data analysis module (840) compares both intra-test and inter-test data (when available) as well as comparative analysis against corrected/adjusted baseline reference data that is coupled based on the degree of association (e.g. the physical subject's parameters of weight, age, height, biological gender, diagnostic code, etc.) and such other inputs collected in (720). The data analysis is based on calculating a number of factors including the rates of instability developed and measuring across the other measurement dimensions to allow data calculations of model convergence and test correlation. It is possible in the data analysis that a measurement previously accepted in (770) can be found non-conforming and this would be reported. It would be dependent upon the context of the testing/screening protocol to determine if a test or series of tests might need to be repeated based on this data analysis performed in (840).

Upon processing the data the results are conveyed to a data reporting module (850) to present the resulting test information in a readable format. This includes a graphical interface to a data display module (750) on the computer work station and conveys the information for storage—including creating/appending to a longitudinal subject data record via a computer network (400) to a host computer (600) which maintains all test results across all testing systems. This data can be further anonymously post processed for continuous ongoing clinical research and continuous refinement of the correcting parameters, refinement of the testing protocols and other research purposes. The data storage also maintains a history of the data performance of the individual force measurement input devices and platforms to include error reporting to determine and predict potential failures and the need to replace input platforms that are non-conforming. This provides a continuous quality check for all the measurement deployments, and thus maintains a consistent and high quality result across all installations.

Optionally, a result can be printed out in (870) to provide the test subject and test provider a paper copy of the subject's test results. This information is the same information that is provided to the data display and the data storage. Optionally this report can be communicated via other media such as a digital memory card (such as an SD Card or other persistent removable memory device) or to other portable electronic devices such as a smart phone.

The system continuously performs measurement “cross checking” in a data validation stage (760, 770) that provides for inherent value confirmations and improves the data correction. This process uses both statistical normalization (standard deviations, etc.) as well as digital filtering techniques (such as applied through Kalman filtering, reverse Kalman filtering, hidden Markov Models and other algorithmic computational techniques both individually and combined) to maximize and improve overall measurement integrity.

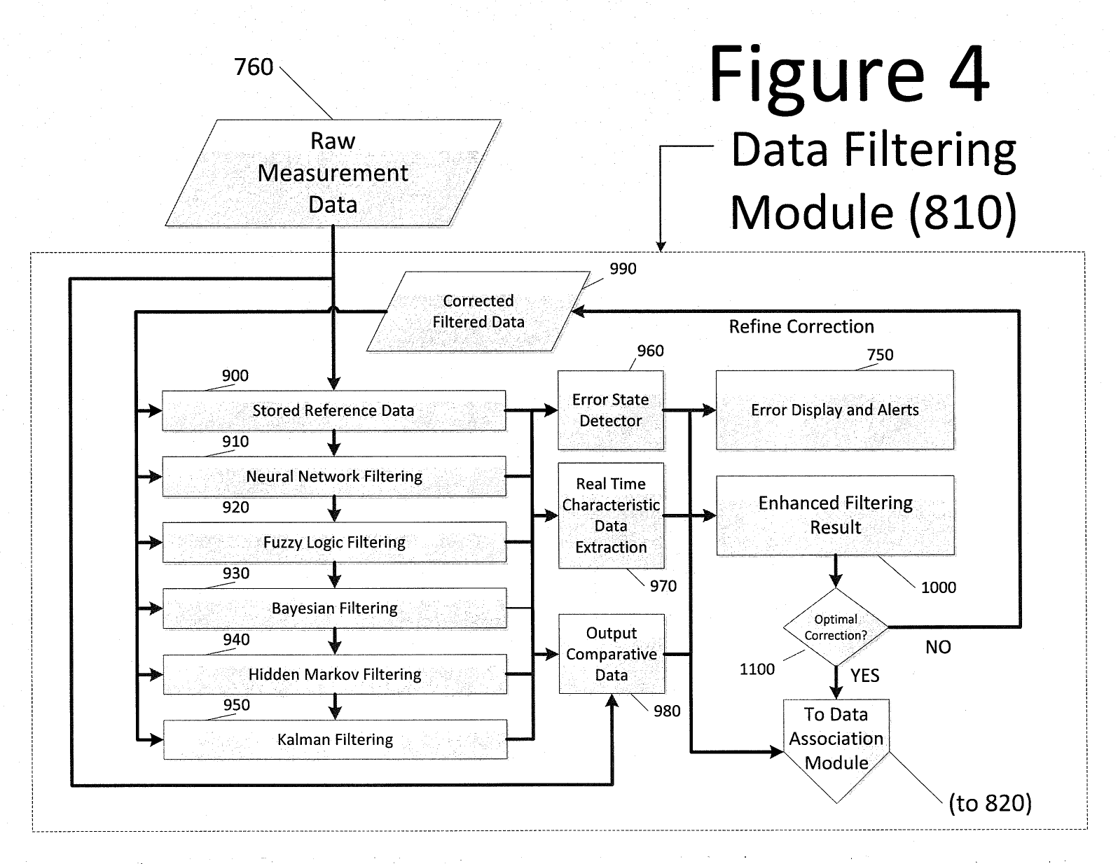

FIG. 4 illustrates an exemplar detail of the data filtering module (810) applying multiple data correction mathematical algorithms to seek optimal corrections and means in both selective sequential and parallel computation of the raw input measurements to improve the data result. This includes evaluating maxima and minima and applying against both predicted inputs and comparatives to other measured maxima. Secondarily, the error and filtering analysis will provide relative comparison of candidate filtering and data improvement methods to determine the best fit (expected v. measured) of the combined data measurements. For example if there was a maxima measured in one test that would be predicted and applied to a second input measurement test this would be evaluated to understand if said correction would be applicable to be applied, and if so, using which mathematical method or methods for seeking the most effective correction.

The raw measurement data (760) is presented to the processing modules for evaluation. In a simple comparison (900) against stored reference data is to determine if the data conforms to an expected range (e.g. if there was a null measurement of a highly unexpected measurement, such as applied when an incorrect test was performed). This would result in a setting a flag in the Error State Detector (960) such that there was an incorrect or inaccurate measurement. The Error would be queued for notification in the Error Display and Alerts (750) function. In parallel or via selective determination, one or multiple filtering methods would be applied to the raw data, including the reference data to be evaluated including Neural Network filtering (910), Fuzzy Logic Filtering (920), Bayesian filtering (930), Hidden Markov Model (940) and Kalman (950), which also could include reverse Kalman filtering as well. The calculated output results from the data filtering are communicated to a real-time characteristic data extraction module (970) which isolates the specific data components of the resulting measurement such as force onset, force maxima, force minima, acceleration moment, variation and time to return to baseline stability and other measures. The characteristic data extraction evaluates both the reference data against the filtered results and provides correlated evaluation of the parametric features. Concurrently, each of the filtered data elements is compared (980) against the last corrected filtered data (990) across the filtered results to determine if the result can be further refined through continuing to apply filtering data correction by comparison of the enhanced filtering result (1000) to prior results. This evaluation also compares against stored reference data (900) as well as the comparative data output (980) which also evaluates against the raw measurement data (760). The result is an evaluation of both the individual and combined adaptive filtering correction results of the data over a time series which provides a way to calculate a predictive value which improves the underlying measurement accuracy. In the optimal correction decision module (1100) the comparative evaluation of the last result to the latest result coupled with the output comparative data (980) is compared to determine if any differences in the result through additional filtering will measurably improve the correction. This direct correction and as well as reverse prediction techniques can be recursively applied across one or more filtering methods to goal-seek the optimal predicted correction. If in the comparison (1100) there is negligible change from the prior result, e.g. the data has converged across the optimal filtering and data correction process, the corrected data is then sent to the association module (820) along with the last output comparative data result (980) along with the characteristic extracted data (970) as well as any error condition (960). When the data residual is minimized this equates to the minimal prediction error.

A priori, based on the type of measurement and test condition, there is no conclusive assertion on which single or combination of filtering, or set of filters as applied will result in calculating the most reliable measurement. For this reason, both the comparative data, including each of the raw datum along with the data characteristic extracted is communicated to the data association module (820) to allow longitudinal improvement of both the reference data as well as the ability to introduce new measures and tests that may be subsequently introduced. The data analysis module (840) develops not only the output analysis which is subsequently reported (850) but also maintains a record of which filtering methods applied for which tests based on the context of the unique underlying determining parameters. This can be used to refine the precision of the solution embodiment, as well as potentially, based on the accuracy of the predictive methods, synthetically determine certain test measurement values solely by calculation, thus reducing the actual number of measurements needed. This allows the evaluation of the tests that need to be administered (730) to be potentially dynamically computed based on the user inputs and test parameters (720).

The foregoing description, for purpose of explanation, has been described with reference to specific embodiments. The illustrative discussions above, however, are not intended to be exhaustive or to limit the invention to the precise forms disclosed. Many modifications and variations are possible in view of the above teachings. The embodiments were chosen and described in order to explain the principles of the invention and its practical applications, to thereby enable others skilled in the art to best utilize the invention and various embodiments with various modifications as are suited to the particular use contemplated.

What I the inventor hereby claims:

1. An integrated system and method for discrete and longitudinal monitoring of multiple properties of neuromuscular function based on measurements of force displacement that includes a self-calibrating method of measurement of a set of at least one parametric acquisition using analysis parameters based on:
a. Identifying the measurement platform and subject,
b. Associating data and information about the subject,
c. Determining the tests to be performed,
d. Performing a set of at least one force measurement test,
e. Evaluating the measurement data,
f. Selectively providing data filtering and data correction for discrete measurement improvement,
g. Selectively processing measurement data against a set of at least one reference,
h. Providing combined data analysis against a set of at least one reference,
i. Storing said results, and
j. Displaying said results.

2. The invention of claim 1, wherein said system comprises at least one measurement device such as a force measurement platform.

3. The invention of claim 1, wherein said analysis derives an indication from a series of measurements and inputs to determine the appropriate types of measurements and associated tests.

4. The invention of claim 1, wherein said sensor platform comprises detection of one or more measurements evaluated by a computer that validates the expected conformance of the acquired data as suitable within programmable acceptable range limits from which a determination can be made that the acquired data is acceptable or not acceptable for processing and evaluation.

5. The invention of claim 1, wherein said sensor platform comprises multiple force platforms such as to measure individual limbs simultaneously.

6. The invention of claim 1 wherein said measurements are further defined by mathematical algorithm processing to compute improved analytic results to provide corrected values across input measurements.

7. The invention of claim 1 is further defined by one or more mathematical algorithms to compute improved analytical results based on applying Kalman filtering techniques.

8. The invention of claim 1 is further defined by one or more mathematical algorithms to compute improved analytical results based on applying Bayesian filtering techniques.

9. The invention of claim 1 is further defined by one or more mathematical algorithms to compute improved analytical results based on applying hidden-Markov filtering techniques.

10. The invention of claim 1 is further defined by one or more mathematical algorithms to compute improved analytical results based on applying fuzzy-logic analysis techniques.

11. The invention of claim 1 is further defined by one or more mathematical algorithms to compute improved analytical results based on applying neural-network analysis techniques.

12. The invention of claim 1 allows for multiple data comparisons to be simultaneously or sequentially analyzed comprising of a set of at least one of the measurement characteristics both on derived and measured data.

13. The invention of claim 1 is further defined to provide for multiple data comparisons based on prior analytic measurements over time.

14. The invention of claim 1 is further defined to provide for multiple data comparisons based on identified similar populations.

15. The invention of claim 1 is further defined to compare calculated comparative expected values to measured values to detect longitudinal changes over time.

16. The invention of claim 1 is substantially facilitated by comparing and adjusting expected baseline data such as predicted calculated changes in performance and resulting measurements correlated due to age.

17. The invention of claim 1 is substantially facilitated by comparing and adjusting expected baseline data such as predicted calculated changes in performance and resulting measurements correlated due to confirmed medical diagnosis.

18. The invention of claim 1 is substantially facilitated by comparing and adjusting expected baseline data such as predicted calculated changes in performance and resulting measurements correlated due to biological gender.

19. The invention of claim 1 includes the method of applying both direct measurement and predicted calculated values for tracking longitudinal changes.

20. The invention of claim 1 is further defined to include calculating anticipated or expected divergence or convergence across measured and calculated values to detect unexpected performance results.

21. The invention in claim 1 allows for the ability to add or modify detection criteria in a standard fashion.

22. The invention of claim 1 allows for algorithmic filtering using multiple sensor inputs to provide corrected values across measurement domains.

23. The invention of claim 1 is further defined by the mathematical algorithm to processing sensor input to compute improved analytic results based on Kalman Filtering techniques across longitudinal changes.

24. The invention of claim 1 is further defined by the mathematical algorithm to processing multi-modal sensor input to compute improved analytic results based on Bayesian analytic techniques across longitudinal changes.

25. The invention of claim 1 is further defined by the mathematical algorithm to processing sensor inputs to compute improved analytic results based on hidden-Markov Filtering techniques across longitudinal changes.

26. The invention of claim 1 is further defined by the mathematical algorithm to processing sensor input to compute improved analytic results based on fuzzy logic analysis techniques across longitudinal changes.

27. The invention of claim 1 is further defined by the mathematical algorithm to processing sensor input to compute improved analytic results based on neural network analysis techniques across longitudinal changes.

28. The invention of claim 1 further allows for several types of data comparison to be analyzed and calculated in parallel.

29. The invention of claim 1 is further defined through calculating expected measurement results based on a time series of a set of at least one performance measurements as adjusted for factors including age, gender, diagnosis, and other factors.

30. The invention of claim 1 includes the method of applying both direct and calculated values for tracking and calculating the time series expected rates of change versus observed rates of change of any single or multiple sensing dimensions.

31. The invention of claim 1 is further defined to include calculating the expected divergence or convergence across multiple sensor time series data of anticipated and expected measured value changes versus unexpected changes.

32. The invention of claim 1 is further defined to include continuously comparative calculations to improve testing protocols and filtering techniques.

33. The invention of claim 1 is further defined to include continuously monitoring force measurement input devices to detect potential failures or performance deviations to indicate maintenance or replacement of measurement input device may be needed.

34. A computer implemented method for discrete and longitudinal monitoring of multiple properties of neuromuscular function based on measurements of force displacement that includes a self-calibrating method of measurement of a set of at least one parametric acquisition using analysis parameters based on:
a. Identifying the measurement platform and subject,
b. Associating data and information about the subject,
c. Determining the tests to be performed,
d. Performing a set of at least one force measurement test,
e. Evaluating the measurement data,
f. Selectively providing data filtering and data correction for discrete measurement improvement,
g. Selectively processing measurement data against a set of at least one reference,
h. Providing combined data analysis against a set of at least one reference,
i. Storing said results, and
j. Displaying said results.
